(19) United States
(12) Patent Application Publication
(10) Pub. No.: US 2008/0131508 A1
(43) Pub. Date: Jun. 5, 2008

(54) Oral transmucosal nicotine dosage form

(75) Inventors: Vikas Agarwal, Plymouth, MN (US); Brian I. Hague, Salt Lake City, UT (US); Rajendra K. Khankari, Maple Grove, MN (US)

Correspondence Address: CEPHALON, INC., 41 MOORES ROAD, PO BOX 4011, FRAZER, PA, 19355, US

(73) Assignees: Cephalon, Inc, Frazer, PA (US); CIMA LABS INC., Eden Prairie, MN (US)

(21) Appl. No.: 11/986,097

(22) Filed: Nov. 20, 2007

Related U.S. Application Data
(60) Provisional application No. 60/872,177, filed on Dec. 1, 2006.
(60) Provisional application No. 60/872,125, filed on Dec. 1, 2006.

(51) Int. Cl. A61K 9/46; A61K 31/465; A61P 25/30

(52) U.S. Cl. 424/466; 514/343

(57) ABSTRACT

The invention described herein relates to an oral transmucosal solid dosage form useful in treating nicotine addiction or as a nicotine substitute or replacement. By virtue of the formulation in combination with nicotine, the invention transmucosally delivers an effective amount of nicotine to the recipient while permitting the accomplishing of such, and manufacture of such, using a relatively small, convenient and orally comfortable dosage form (e.g., tablet) size.

### RELATED APPLICATION DATA

This application claims benefit of priority to U.S. provisional application Nos. 60/872,177 and 60/872,125, both of which were filed on Dec. 1, 2006.

### FIELD OF THE INVENTION

The invention relates to the field of pharmaceutical dosage forms and methods of treatment. In particular, the invention pertains to an oral transmucosal dosage form containing nicotine or nicotine derivative, and methods of treating nicotine withdrawal therewith.

### BACKGROUND OF THE INVENTION

A wide variety of nicotine cessation products and therapies are known. Such products include lozenges, gums, transdermal patches, and the like. Lozenges and gums provide oral delivery of nicotine, whereas transdermal patch treatments deliver nicotine through the wearer's skin. These systems are founded on the premise that successful smoking cessation programs require control of the craving episodes associated with nicotine addiction. One example of an oral lozenge-type product is available commercially as COMMIT® (Glaxo-Smithkline, Philadelphia, Pa.). These lozenges are relatively bulky and large in size, and are intended to be swished around within the mouth of the user. Thus, a significant amount of the nicotine can be swallowed, and the delivery of nicotine can be delayed. Further, as with oral gastrointestinal route nicotine treatments, nicotine ingested is subject to first pass metabolism which further reduces systemic delivery of the desired effective amount of active.

Oral transmucosal delivery of nicotine is known. Passive introduction of nicotine to mucosal tissue, such as that introduced by NICORETTE® gum, can deliver amounts of nicotine transmucosally. One problem, however, is that the administration mechanism or dosage form is heavily commingled with the recipient's saliva, and the active ingredient gets “diluted” within the recipient's oral cavity. Further, the systemic receipt of the active can be significantly delayed.

Delayed delivery of nicotine to a recipient experiencing a nicotine “craving” to rapidly offset the craving can often determine the success or failure of a nicotine cessation product or program. In order to address a craving episode promptly, it is desirable to achieve a front-loaded nicotine delivery to the user's system.

One oral dosage form specifically formulated for effective oral transmucosal absorption of certain opiates, such as fentanyl, has been developed under the brand name FENTORA® utilizing the ORAVESCENT® technology (available from CIMA LABS INC., Eden Prairie, Minn.). This technology has been described in U.S. Pat. Nos. 6,200,604 and 6,974,590, for example, as well as U.S. Published Patent Application Nos. 2005/0169989 (Ser. No. 1/026,132 filed Dec. 30, 2004); 2005/0142197 (Ser. No. 1/026,327 filed Dec. 30, 2004); 2005/0142198 (Ser. No. 1/027,353 filed Dec. 30, 2004); and 2005/0163838 (Ser. No. 11/026,759 filed Dec. 30, 2004)—all of which are pending and incorporated herein by reference. This particular technology uses an excipient formulation containing a pH adjusting substance and effervescent couple to facilitate transmucosal transport of active ingredient fentanyl citrate.

There exists a need in the field of nicotine cessation or replacement therapy and products for an improved oral transmucosal dosage form that effectively and rapidly deliver nicotine to a recipient. There is further need for a nicotine composition that permits preparation of relatively smaller sized dosage forms, while delivering comparably effective amount of active nicotine to the recipient.

### SUMMARY OF THE INVENTION

The invention provides an oral transmucosal nicotine dosage form that utilizes effervescence and localized pH adjustment to effectively and rapidly deliver a therapeutically effective amount of nicotine (or nicotine derivative) to a recipient. It has been discovered that nicotine can also be effectively delivered transmucosally using an improved effervescent solid dosage form intended for static resident placement adjacent the recipient's mucosal tissue. It has also been discovered that because of its enhanced bioavailability, smaller tablets can be manufactured to deliver effective amounts of nicotine, thereby permitting more convenient packaging, cost effective manufacturing, and a more comfortable oral administration experience. Thus, effective concentrations of nicotine can be delivered transmucosally and rapidly avoiding first pass metabolism using a relatively smaller dosage form as compared to traditional oral nicotine dosage forms.

The invention provides a solid oral transmucosal dosage form comprising the following ingredients: nicotine or nicotine derivative as an active ingredient; an effervescent couple; and a pH adjusting substance; the dosage form being formulated for static resident placement within a recipient's oral cavity for transmucosal delivery of the nicotine or nicotine derivative across said recipient's oral mucosal tissue. In a preferred embodiment, the dosage form is in the form of a buccal tablet.

As a result of the enhanced transmucosal transport afforded by the formulation prepared in accordance with the invention, a smaller amount of active nicotine in the formulation can effectively deliver a relatively large amount of nicotine to the recipient (Cₘₐₓ) in a relatively short time period (Tₘₐₓ). One of the important advantages associated with the instant invention is that by virtue of the combination of ingredients, a given effective nicotine dosage can be achieved with a relatively smaller tablet weight or size because of the achievable earlier bioavailability (e.g., Cmax of about 61 ng/ml as soon as about Tmax 13 minutes after placement in the oral cavity in a mammal) afforded by the invention is comparable to existing commercial products despite the relatively small tablet size (e.g., approximately 5/16″ in one embodiment). Because of the comparable bioavailability of nicotine when prepared according to the invention, a relatively smaller, more convenient tablet size can be manufactured which delivers same effective amount of nicotine to the user and can achieve the same therapeutic effectiveness as compared to larger lozenge-type products. Put another way, the invention permits the manufacture of a relatively small tablet that achieves comparable bioavailability of active nicotine, or therapeutic effect per tablet size.

The dose of nicotine or nicotine derivative contained in the composition of the invention can be adjusted to achieve the desired Cmax. The compositions formulated for the canine studies were formulated to deliver a Cmax of about 61 ng/ml. It will be understood, however, that compositions can be prepared according to the invention which accomplish a variable Cmax based on desired effect. For nicotine substitution purposes and smoking cessation purposes, the composition can be formulated to deliver a Cmax ranging from about 3 ng/ml to about 70 ng/ml (and a Tmax of about 3 minutes to about 40 minutes), preferably 7 ng/ml to about 50 ng/ml (and a Tmax of about 4 minutes to about 30 minutes). Nevertheless, it is believed that when administered to humans, to achieve a bioavailability appropriate to provide an amount of nicotine sufficient to address a craving episode and a level that would still avoid undesirable side effects such as nausea, a Cmax ranging from about 10 ng/ml to about 25 ng/ml is most preferred (and Tmax of about 5 minutes to about 20 minutes). This estimation is based on the content as described in Hukkanen et al., entitled “Metabolism and Disposition Kinetics of Nicotine”, Pharmacological Reviews, Vol. 57, No. 1, pages 79-115 (2005) in conjunction with the findings of the canine bioavailability study set forth herein below.

The invention provides a solid oral transmucosal dosage form comprising the following ingredients: nicotine or nicotine derivative as an active ingredient; an effervescent couple; and a pH adjusting substance; wherein the dosage form is formulated for resident placement within a recipient's oral cavity and for transmucosal delivery of said nicotine or nicotine derivative across the recipient's oral mucosal tissue. In a preferred embodiment, the dosage form is a buccal tablet.

The invention further provides a method for treating nicotine addiction in a recipient desiring such treatment, said method comprising: a) providing to the recipient a solid oral dosage form comprising the following ingredients: nicotine or nicotine derivative as an active ingredient; an effervescent couple; and a pH adjusting substance; wherein the dosage form is formulated for resident placement within a recipient's oral cavity and for transmucosal delivery of said nicotine or nicotine derivative across the recipient's oral mucosal tissue; b) positioning the dosage form within the recipient's oral cavity adjacent to oral mucosal tissue; and c) permitting said dosage form to reside in such position for a period of time sufficient to permit the nicotine or nicotine derivative to transport across the oral mucosal tissue. In one embodiment, the method can provide a Cₘₐₓ from about 3 ng/ml to about 70 ng/ml at a Tₘₐₓ of about 3 minutes to about 40 minutes to the recipient. Alternatively or as a further embodiment, the invention also provides a method of nicotine substitute comprising providing a recipient desiring such substitution a dosage form containing nicotine prepared according to the invention.

The invention further provides an oral transmucosal nicotine delivery system comprising a solid oral transmucosal dosage form comprising: nicotine or nicotine derivative as the active ingredient; an effervescent couple; and a pH adjusting substance; the dosage form being formulated for placement within a recipient's oral cavity for transmucosal delivery of nicotine or nicotine derivative across the recipient's oral transmucosal tissue; in combination with a holder; wherein the dosage form is coupled to an end of the holder. In one embodiment, the holder is a hand-held stick.

These and other features and advantages of the invention will become apparent from the following disclosure.

### BRIEF DESCRIPTION OF THE DRAWINGS

The following figures further illustrate the invention, and none are intended to imply a necessary limitation to the claimed invention.

FIG. 1 is a chart showing comparative mean plasma concentrations versus time for various solid nicotine dosage form formulations.

FIG. 2 is an illustration of a transmucosal nicotine delivery system with a dosage form on holder, according to one embodiment of the invention.

### DETAILED DESCRIPTION OF THE INVENTION

As used herein, the phrase “oral transmucosal,” within the context of drug delivery and absorption, is meant to refer to the pre-peristaltic stage of uptake of the drug via one or more of the mucosal tissue types associated with the oral cavity, e.g., sublingual, buccal, gingival, palatal, esophageal regions of oromucosal tissue. More specifically, what is intended by the phrase is that the primary delivery route of the active ingredient occurs through the mucosal tissue of the oral cavity.

As used herein, the term “about” refers to a range of values from ±10% of a specified value, and functional equivalents thereof unless otherwise specifically precluded. For example, the phrase “about 50 mg” includes ±10% of 50, or from 45 mg to 55 mg.

As used herein, the term “therapeutically effective amount” is meant to refer to the amount determined to be required to produce the physiological effect intended and associated with the given active ingredient, as measured according to established pharmacokinetic methods and techniques, for the given administration route.

As used herein, the phrase “oral dosage form”, when used in the general sense, includes orally disintegrable/dissolvable tablets, capsules, caplets, gels, creams, films, sprays, and the like. Within the specific context of the instant invention, the oral dosage form of the invention refers to the pharmaceutical composition of the invention as a solid oral dosage form comprising a nicotine or nicotine derivative, accompanied by an excipient formulation which facilitates and enhances oral transmucosal absorption of the active ingredient as defined by the invention.

As used herein, the term “substantially”, unless otherwise defined, is meant to refer to a specific property, characteristic or variable that meets the stated criteria in such measure that one skilled in the art would understand that the benefit to be achieved, or the condition or property desired, is met.

The compositions of the invention are discussed herein within a general context of being “formulated for resident placement within a recipient's oral cavity for transmucosal delivery of said nicotine or nicotine derivative across said recipient's oral mucosal tissue.” This phrase, and like phrases made herein, are meant to indicate that by virtue of the collective combination of ingredients, their individual and combined functionalities, and the techniques used to prepare the dosage form, provide a dosage form that affords delivery of the active ingredient (nicotine) across the recipient's mucosal tissue when placed adjacent thereto for a period of time sufficient to permit such transport.

Compositions prepared according to the invention contain nicotine or a nicotine derivative as an active pharmaceutical ingredient. Suitable nicotine derivatives that can be used include pharmaceutically acceptable resin complexes and pharmaceutically acceptable salts of nicotine. Suitable nicotine derivatives include, but are not limited to, nicotine polacrilex and nicotine bitartrate. For therapeutic effect for smoking cessation purposes (i.e., delivery of nicotine in an amount sufficient to address the craving episode), the absorbed amount needed can vary. As a result in part of the combination of formulation ingredients used to prepare the composition of the invention, however, a relatively small amount of nicotine per dosage unit is needed in order to achieve the desired therapeutic effect faster as a result of the Cₘₐₓ and Tₘₐₓ pharmacokinetic parameters associated with the formulation in the form of a resident tablet.

For compositions prepared in accordance with the invention, dose amounts of nicotine that can be used can range from about 0.5 mg to about 4.0 mg, but are variable based on the desired therapy, results or effect. Within a smoking cessation context, dosage forms prepared according to the invention can be administered with a frequency sufficient to achieve a total daily dosage amount of up to about 60 mg/day. The total daily dosage of nicotine or nicotine derivative desired will vary according to the individual's specific therapeutic, cessation or substitution needs, preferences or requirements.

Generally, nicotine compositions prepared according to the invention comprise an effervescent couple to function as an absorption enhancer, preferably in combination with a pH adjusting substance. A variety of effervescent couples can be used in the invention. For example, the effervescent couples described in U.S. Pat. No. 5,178,878 and U.S. Pat. No. 5,503,846 can be used—the entire texts of which are incorporated herein by reference. In general, effervescent couples for the invention include those that are water- or saliva-activated materials usually kept in anhydrous state with little or no absorbed moisture, or in a stable hydrated form. Suitable effervescent couples can comprise at least one food grade acid and at least one food grade reactive base, which can be a carbonate or bicarbonate.

Suitable acids for use in the effervescent composition include food grade acids, acid anhydrides and acid salts. Food grade acids include, but are not limited to, citric acid, tartaric acid, malic acid, fumaric acid, adipic acid, ascorbic acid and succinic acid, and acid anhydrides or salts thereof. Salts used can be food grade sodium, potassium and calcium salts, e.g., sodium dihydrogen phosphate and disodium hydrogen phosphate, and acid citrate salts and disodium acid sulfate. Preferably, citric acid is used.

Bases that can be used in accordance with the invention include, but are not limited to, sodium bicarbonate, potassium bicarbonate, and the like. Sodium carbonate, potassium carbonate, magnesium carbonate and the like can also be used to the extent they are used as part of the effervescent couple, but can also be used as a pH adjusting substance in combination with the effervescent couple.

The amount of effervescent couple component useful in accordance with the invention is an effective amount and is determined based on properties other than those which would be necessary to achieve disintegration of a tablet in the mouth. Instead, effervescence is used in the invention as a basis for enhancing transmission of the active ingredient across mucosal membranes via buccal, sublingual or gingival administration in the oral cavity. Accordingly, the amount of effervescent couple should range between about 5 to about 85 percent, more preferably between about 15 and 60 percent, even more preferably between about 30 and 45 percent, and most preferably between about 35 and 40 percent, based on total formulation weight. Of course, the relative proportion of acid and base will depend upon the specific ingredients, e.g., whether the acid is mono-, di- or tri-protic, relative molecular weights, etc.

Most preferably, the pH adjusting substance is an ingredient in addition to and other than one of the components of the effervescent couple. A compound that is susceptible to changes in ionization state can be administered by effecting the proper conditions for its dissolution and transmission across tissues within the oral cavity. If the ideal conditions for a particular drug are basic, the addition of sufficient excess of a suitable strong acid as part of either the effervescent composition or the pH adjusting substance may not be indicated. The selection of another pH adjusting substance, for example anhydrous sodium carbonate, which functions separate and apart from the effervescent couple would be preferred.

Various pH adjusting substances can be used to provide further permeation enhancement of the active ingredient. The selection of the appropriate pH adjusting substance will depend on the drug to be administered and, in particular, to the pH at which the drug is ionized or unionized, and whether the ionized form or unionized form facilitates transmission across the mucosa.

In one embodiment, the pH adjusting substance is any substance that is capable of adjusting the localized pH to promote transport across the mucosa in amounts which will result in a pH generally ranging from about 3 to about 10, more preferably between about 4 to about 9. The pH is the “localized pH” at the microenvironment at the surface contact area of the oral mucosa and the dosage form (or portions of it as it disintegrates and/or dissolves) once placed in the mouth of the recipient.

In general, the localized pH can be determined by initially characterizing the dynamic pH changes displayed by the tablets using in vitro pH measurement. The method consists of using 0.5-10 ml phosphate buffered saline in an appropriately sized test tube or other similar vessel. One liter volume of buffered saline solution can be prepared by dissolving 9.0 g sodium chloride, 0.6 g sodium phosphate monobasic monohydrate and 0.78 g of sodium phosphate dibasic (anhydrous) in about 1000 ml of deionized water, and adjusting the pH to 7.0±0.05 at room temperature by adding 1 N sodium hydroxide with stirring. The adjustment should require about 0.5 ml. The amount of media used depends on the tablet size and dosage. For example, a volume of 2 ml can be used for a tablet weighing 200 mg. Immediately upon contact with the media, the pH profile of the solution is monitored as a function of time, using a micro-combination pH electrode.

Preferably, the materials which can be used as pH adjusting substances in accordance with the present invention include carbonate, bicarbonate, phosphate, hydrogen phosphate and dihydrogen phosphate. Suitable carbonates include sodium carbonate, potassium carbonate or calcium carbonate. Suitable phosphates include calcium phosphate or sodium phosphate. Most preferred for use as a pH adjusting substance is sodium carbonate. Also preferred are pH adjusting substances which, when provided in suitable amount, can provide a change in localized pH of at least about 0.5 pH units, more preferably 1.0 pH units, even more preferably about 2.0 pH units when compared to an otherwise identical formulation without the pH adjusting substance.

The amount of pH adjusting substance can vary with the type of pH adjusting substance used, amount of excess acid or base from the effervescent couple, the nature of remaining ingredients, and the active ingredient. Preferably, the amount of pH adjusting substance can vary from about 0.5 to about 25 percent, more preferably between about 2 to about 20 percent, even more preferably between about 5 to about 15 percent, most preferably between about 7 and about 12 percent by weight of the total formulation weight.

When the composition is in the solid dosage form of a tablet, one embodiment of the composition of the invention further comprises a filler, disintegrant, or lubricant, and combinations thereof. Any filler or any amount of a filler can be used as long as the resulting dosage forms achieve the results described herein. Most preferred amongst the fillers are sugar and sugar alcohols, and these may include non-direct compression and direct compression fillers. Non-direct compression fillers generally, at least when formulated, have flow and/or compression characteristics which make them impractical for use in high speed tableting process without augmentation or adjustment. For example, a formulation may not flow sufficiently well and therefore, a glidant such as silicon dioxide may need to be added.

Direct compression fillers, by contrast, do not require similar allowances. They generally have compressibility and flowability characteristics which allow them to be used directly. It is noted that, depending upon the method by which the formulations are made, non-direct compression fillers may be imparted with the properties of direct compression fillers. The reverse is also true. As a general matter, non-direct compression fillers tend to have relatively smaller particle size when compared to direct compression fillers. However, certain fillers such as spray dried mannitol have relatively smaller particle sizes and yet are often directly compressible, depending on how they are further processed. There are also relatively large non-direct compression fillers as well.

Suitable fillers for use with the invention include, but are not limited to, mannitol, lactose, sorbitol, dextrose, sucrose, xylitol and glucose, to the extent that they can provide the results described herein. Preferred for use as the filler is spray dried mannitol. The amount of filler used can range from about 10 to about 80 percent, more preferably from about 25 to about 80 percent, most preferably from about 25 to about 60 percent by weight of the formulation.

Disintegrants can also be used in the composition of the invention. Disintegrants can permit dosage reduction and/or increase the ratio of Cmax and dose. Disintegrants can include binders that also have disintegrant properties. Suitable disintegrants include, but are not limited to, microcrystalline cellulose, cross-linked polyvinyl pyrrolidone (PVP-XL), sodium starch glycolate, croscarmellose sodium, cross-linked hydroxypropyl cellulose, and the like. Selection of the disintegrant can depend upon whether or not, within a given system, the results described can be obtained with its use. Most preferred for use as a disintegrant is a starch glycolate, more preferably sodium starch glycolate.

The amount of disintegrant can vary according to factors such as dosage form size, nature and amount of other ingredients, and the like. Generally, the amount of disintegrant can range from about 0.25% to about 20% by weight of the final formulation, preferably between about 0.5% and about 15% w/w, more preferably between about 0.5% and about 10% w/w, most preferably between about 1% and about 8% by weight—based on the weight of the finished formulation.

The invention can further comprise a tableting or ejection lubricant. Suitable lubricants include, but are not limited to, magnesium stearate, stearic acid, calcium stearate, and combinations thereof. Preferred for use as the lubricant is magnesium stearate. Generally, the amount of lubricant should generally be less than 1% of the formulation by weight—ideally less than about 0.5%. In the case of magnesium stearate, however, the amount can be greater than about 1.0% provided the amount does not adversely affect the desired properties of the resulting dosage form, preferably greater than 1.5% and more preferably between about 1.5% and about 3%. When magnesium stearate is used, most preferably the amount is about 2% by weight.

The composition of the invention can include other conventional excipients in generally known amounts provided they do not significantly detract from the advantageous attributes afforded by the invention. Such additional excipients can include, but are not limited to, binders, sweeteners, coloring agents, flavoring agents, glidants, lubricants, disintegrants, preservatives, and the like.

The composition of the invention can be prepared as a solid oral transmucosal dosage form, e.g., tablet. Effervescent tablets prepared in accordance with the invention can be relatively robust or soft. For example, tablets containing the composition of the invention can generally be prepared according to the manufacturing methods described in U.S. Pat. No. 5,178,878, the text of which is incorporated herein by reference. When prepared according to this technique, the dosage form can have a hardness of less than about 15 Newtons, but the active ingredient need not necessarily be, and preferably is not, coated with a protective material. When soft friable tablets are produced, they can be advantageously packaged in blister packs such as those described in U.S. Pat. No. 6,155,423. Alternatively, robust dosage forms with a hardness of greater than about 15 Newtons can be manufactured according to the process described in U.S. Pat. No. 6,024,981. Further, the degree of state of powder, e.g., reproducibility and/or consistency of particle size, can affect results.

One of the important advantages associated with the instant invention is that by virtue of the collective combination of ingredients, their functionality, and their manufacturing process, the compositions of the invention are formulated for transmucosal absorption of the active ingredient in the form of a stationary or resident dosage form, e.g., tablet, that can be placed in the recipient's oral cavity. By preparing a dosage form according to the invention, a given effective nicotine dosage can be achieved with a relatively small, orally comfortable, tablet weight or size because of the achievable earlier bioavailability and pharmacokinetic parameters.

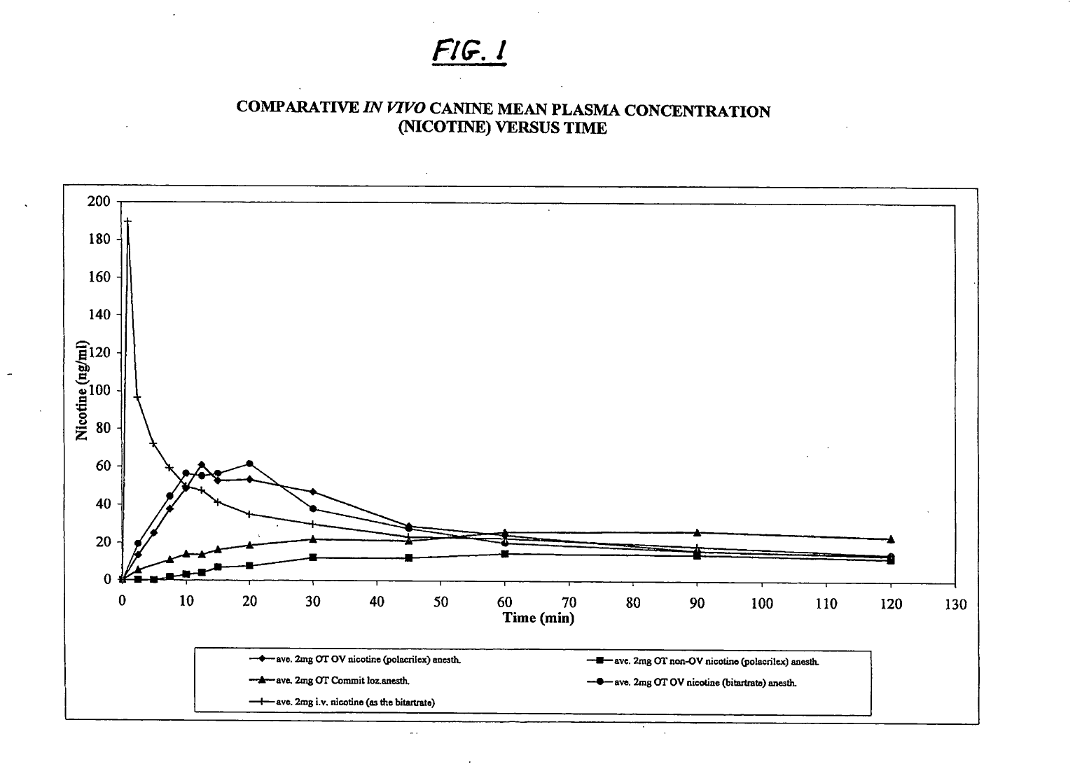

In one embodiment and as described in Example 1, Table 1 in conjunction with the corresponding data in FIG. 1, a 2 mg nicotine derivative 200 mg 5/16″ diameter tablet prepared according to the invention can deliver a serum nicotine concentration Cmax of about 61 ng/ml (Cmax 61.33 ng/ml) as soon as about 13 minutes (tmax 13.33 minutes) after placement in the oral cavity of a dog) afforded by the invention is comparable to existing commercial products despite the relatively small tablet size approximately 5/16″ diameter in one embodiment. In another embodiment using nicotine bitartrate dihydrate and similar dose and tablet size, a Cmax of about 65 ng/ml (Cmax 64.67 ng/ml) can be achieved in about 17 minutes (tmax of 17.50 minutes).

As a result, smaller and more convenient packaging systems can be used with, for example, 200 mg tablets having a diameter of about 5/16 inches. Because of the effervescent ingredients, however, it is preferred that the packaging systems used with the invention be those that prevent environmental moisture or humidity from accessing the prepared dosage forms. For instance, blister packs containing the dosage form of the invention can be prepared using conventional techniques and equipment readily available to those skilled in the pharmaceutical packaging field.

The invention also provides a method for treating nicotine addiction in a recipient desiring such treatment, said method comprising:

a) providing to said recipient a solid oral dosage form comprising:
i) nicotine or nicotine derivative as an active ingredient;
ii) effervescent couple; and
iii) pH adjusting substance;
said dosage form being formulated for static resident placement within a recipient's oral cavity for transmucosal delivery of said nicotine or nicotine derivative across said recipient's oral mucosal tissue;
   b) positioning said dosage form within the recipient's oral cavity adjacent to oral mucosal tissue; and
   c) permitting said dosage for to reside in such position for a period of time sufficient to permit the nicotine or nicotine derivative to transport across the oral mucosal tissue.

According to the invention, placement of the dosage form within the oral cavity of the recipient can be adjacent mucosal tissue to permit transmucosal delivery of the nicotine or nicotine derivative. Thus, the dosage form can be placed in a number of locations, including but not limited to, buccally, sublingually, and gingivally. Preferably, the dosage form is placed in the buccal cavity of the recipient. As the invention affords the preparation of relatively small dosage form sizes, several comfortable oral positions for the dosage form are available to the recipient.

The dosage form prepared according to the invention is water-soluble and water-dispersible, and disintegrates upon contact with the recipient's saliva to release the active ingredient. Residence time, and the period of time sufficient to permit the nicotine or nicotine derivative to transport across the mucosal tissue, can vary according to the specific formulation, ingredients selected, and it processing and manufacture technique. In one embodiment, the dosage form can disintegrate in situ within a time period ranging from about 3 minutes to about 10 minutes. Of course, the recipient's behavior relative to the dosage form can also affect disintegration time.

In another aspect, the invention provides a method for replacing or substituting nicotine sources, such as cigarettes and chewing tobacco. The composition and dosage forms prepared according to the invention can, therefore, provide an alternative source for nicotine, which may or may not share the objective of nicotine cessation. According to this embodiment, the composition or dosage form would provide a recipient active nicotine or nicotine derivative without the disadvantages, health risks and/or carcinogens associated with tobacco-derived nicotine intake. Overall, the method steps performed for the method of cessation likewise apply for nicotine source substitution practices. Specifically, this method of nicotine substitution can comprise: providing a dosage form to a recipient desiring a non-tobacco nicotine source, comprising: nicotine or nicotine derivative as an active ingredient; an effervescent couple; a pH adjusting substance; wherein the dosage form is formulated for placement within the recipient's oral cavity for transmucosal delivery of the nicotine or nicotine derivative across the oral mucosa. The recipient can then place the dosage form within the recipient's oral cavity adjacent to recipient's mucosal tissue, and permits said dosage form to reside adjacent said mucosal tissue for a period of time sufficient to deliver nicotine across the mucosal tissue.

### EXAMPLES

### Example 1

### Preparation of Packaged Oral Transmucosal Dosage Form (Tablet) Containing 2 mg Nicotine from Nicotine Polacrilex

A 200 mg solid oral transmucosal tablet was prepared having a nicotine polacrilex potency (15%) effective to deliver 2 mg dose active nicotine. Nicotine polacrilex, mannitol (spray-dried), sodium bicarbonate, citric acid, sodium carbonate and sodium starch glycolate were sieved and blended in a mixer for a predetermined period of time (about 30 minutes). After this mixture was prepared, magnesium stearate was then added to the mixture and blended for about 5 minutes. The resultant mixture was then discharged and compressed on a rotary tablet press thereby forming tablets to defined and predetermined weight (200 mg) and hardness (10 N). The tablets were then sorted and packaged into aluminum-aluminum blister packs. The blending, tableting and blister packing operations were all undertaken in humidity controlled environmental conditions of less than 25 grains of moisture per pound dry air.

The resulting tablet contained the following formulation:

TABLE 1
2 mg Nicotine (from Nicotine Polacrilex) Tablet

| Ingredient: | mg/tablet | % w/w |
|---|---|---|
| Nicotine polacrilex (15%) | 13.33 | 6.67 |
| Mannitol (spray-dried) | 84.67 | 42.33 |
| Sodium bicarbonate | 42.00 | 21.00 |
| Citric acid | 30.00 | 15.00 |
| Sodium carbonate | 20.00 | 10.00 |
| Sodium starch glycolate | 6.00 | 3.00 |
| Magnesium stearate | 4.00 | 2.00 |
| Total: | 200.00 mg | 100.0% |

* Nicotine polacrilex is based on 15% potency and a 2 mg dose of nicotine.

### Example 2

### Preparation of Oral Transmucosal Dosage Form (Tablet) containing 2 mg Nicotine from Nicotine Bitartrate

Using a procedure similar to that described above in Example 3, a 200 mg solid oral transmucosal tablet containing nicotine bitartrate as the active nicotine source was prepared. The formulation appears in the following table:

TABLE 2
2 mg Nicotine (from Nicotine Bitartrate) Tablet

| Ingredient: | mg/tablet | % w/w |
|---|---|---|
| Nicotine bitartrate dihydrate (34%)* | 6.15 | 3.08 |
| Mannitol (spray-dried) | 91.85 | 45.92 |
| Sodium bicarbonate | 42.00 | 21.00 |
| Citric acid | 30.00 | 15.00 |
| Sodium carbonate | 20.00 | 10.00 |
| Sodium starch glycolate | 6.00 | 3.00 |
| Magnesium stearate | 4.00 | 2.00 |
| Total: | 200.00 mg | 100.0% |

*Nicotine bitartrate dihydrate is based on 34% potency and a 2 mg dose of nicotine.

### Example 3

### Comparative 2 mg Nicotine (from Nicotine Polacrilex) Formulation

Using a process similar to that described above in Example 1, a 200 mg nicotine tablet formulation was prepared containing the remaining excipient components preferred for use with the instant invention but absent the effervescent couple and pH adjusting substance ingredients of the invention. The filler ingredient, mannitol, was used to replace the effervescent couple and pH adjusting ingredient amounts in the nicotine polacrilex formulation of Example 1. The resulting formulation is set forth in the following tablet:

TABLE 3
Comparative 2 mg nicotine (from nicotine polacrilex) Formulation

| Ingredient: | mg/tablet | % w/w |
|---|---|---|
| Nicotine polacrilex (15%)* | 13.33 | 6.67 |
| Mannitol (spray-dried) | 176.67 | 88.33 |
| Sodium starch glycolate | 6.00 | 3.00 |
| Magnesium stearate | 4.00 | 2.00 |
| Total: | 200.00 mg | 100.0% |

*Nicotine polacrilex is based on 15% potency and a 2 mg dose.

### Example 4

### Comparative In Vivo Bioavailability Data

Commercial product formulation COMMIT® Lozenge (available from Glaxo Smithkline Beecham), an oral 2 mg nicotine (from nicotine polacrilex) dosage form, was obtained and used in a comparative experiment. The purpose of the experiment was to evaluate bioavailability or PK parameters associated with four formulations (inventive formulations of Table 1 and Table 2, comparator formulation Table 3 (prepared without the ingredients essential to the invention) and the commercial product COMMIT®. The 2 mg nicotine COMMIT lozenge used in the comparison had a lozenge weight of 1225 mg. For purposes of the experiment, the COMMIT lozenge was placed adjacent the mucosa for static positioning to “mimic” a static buccal transmucosal-type dosage form, despite the instructions associated with the product which instruct swishing around within the oral cavity.

Alongside the solid dosage forms used in the experiment, an intravenously-administered solution was also prepared and used in the experiment to use as the basis for calculating theoretical absolute bioavailability of the solid dosage forms. A 5 ml of 1 mg/ml nicotine solution was prepared by dissolving 15.36 mg nicotine bitartrate dihydrate in water added until a total amount of 5 ml was reached. The solution was prepared based on the nicotine bitartrate dihydrate nicotine base:salt ratio of 3.07. Next, 15.36 mg nicotine bitartate dihydrate was weighed into a tared sterile 5 ml vial, into which was added 5 ml sterile water for injection (SWFI). The solution was aspirated into a 5 ml syringe. Onto the syringe tip was placed a 0.2 micron filter, and a 18 gauge needle was placed onto the filter and the solution transferred through the filter/needle assembly into an empty sterile 5 ml vial. The vial was dated to expire within 24 hours.

In the in vivo experiment, the i.v. solution was administered to average 2 mg nicotine bitartrate administration at a rate of 1 ml/min for a period of 2 minutes, which corresponded to the highest oral transmucosal dose tested in solid form. Samples were drawn at zero time and predetermined time intervals set forth in FIG. 1 (see 2 mg i.v. nicotine key). After being drawn, the samples were left to stand for 10 minutes and then centrifuged to provide the serum samples for analysis.

For the bucally administered dosage forms, a 5/16 inch diameter dosage unit was placed in the lower buccal cavity of the canine subject opposite to the side of the mouth that was resting on the surgical table. Then, 100 to 200 μl saliva substitute (sodium chloride/sodium phosphate solution adjusted to pH 7.0 using sodium hydroxide) was instilled at t=0 and every 2.5 minutes until the dissolution of the dosage unit was achieved. The subject's mouth was kept open but not stretched with jaw clamp to avoid stress to the masseter muscle. The mouth was washed and wiped before the experiment began and unclamped at 15 minutes after start time. A zero time sample was drawn before placement of the dosage unit in the buccal cavity, followed by arterial samples of appropriate volume drawn at predetermined time intervals (see FIG. 1). The samples are left to stand 10 minutes before centrifuging and serum analysis. In both the solid dosage unit and intravenous testing samples, a dosage averaging 2 mg nicotine was administered.

Each canine subject was restricted to fluids for 12 hours prior to the study and sedated with propofol before intubation. The i.v. line was inserted into the cephalic vein and followed by Normal Saline infusion at approximately 15 ml/kg (480 ml/hr) for 1 hour, then 5 ml/kg (160 ml/hr) for the remaining time. After i.v. line insertion, the subject is then connected to a closed circuit delivering 2% isoflurane. Alternatively, for conscious sedation subjects, alternatively medetomide HCl was administered. An arterial line was inserted in the femoral artery for collection of the arterial blood samples. For conscious sedation, serum samples were obtained via the cephalic line to avoid discomfort and stress on the subject. Sample volumes were recorded.

After centrifugation and serum analysis, the serum concentrations and bioavailability parameters were calculated. The bioavailability data is set forth in the following table and also plotted in FIG. 1.

TABLE 4
Comparative in vivo Canine Bioavalability Data

| | Ex. 1, Table | Ex. 3, Table | | Ex. 2, Table | |
|---|---|---|---|---|---|
| | 1 | 3 | Commercial | 2 | I.V. |
| | Measurement: | | | | |
| | 2 mg OT | 2 mg OT | | 2 mg OT | |
| | OV | non-OV | 2 mg “OT” | OV | 2 mg i.v. |
| | Nicotine | nicotine | (commercial | Nicotine | nicotine |
| | (polacrilex) | (polacrilex) | lozenge) | (bitartrate) | (bitartrate) |
| Cₘₐₓ (ave. | 61.33 | 15.67 | 28.33 | 64.67 | 189.61 |
| ng/ml) | | | | | |
| tₘₐₓ (ave. | 13.33 | 55.00 | 80.00 | 17.50 | 1.00 |
| ng/ml) | | | | | |
| AUC₀₆₁₂₀ | 3085 | 1377 | 2652 | 3228 | 3424.21 |
| Bioavail. | 92.77 ± 25.93 | 42.92 ± 23.33 | 83.35 ± 12.50 | 101.58 ± 9.98 | — |

n = 3
OT = oral transmucosal
OV = formulated according to the invention with effervescent couple and pH adjusting substance.
Non-OV = formulated outside of the invention, i.e. without effervescent couple and pH adjusting substance.

The results were plotted and shown in FIG. 1. As can be seen from the above data, the tablet formulations prepared according to the invention (the two formulations of Example 1 Table 1 and Example 2 Table 2 appearing as “2 mg OV nicotine (polacrilex)” and “2 mg OV nicotine (bitartrate)” respectively) clearly show a substantially higher Cₘₐₓ and substantially shorter tₘₐₓ as compared to the formulation of Example 3 Table 3 (appearing as “non-OV nicotine”) and comparator product COMMIT. The oral transmucosal dosage forms containing the effervescent and pH adjusting ingredients prepared according to the invention exhibited faster onset action in terms of Cₘₐₓ and tₘₐₓ bioavailability and pharmacokinetics as compared to even the comparator formulation absent the effervescent and pH adjusting ingredients.

Regarding the dosage units prepared according to the invention (Example 1, Table 1 and Example 2, Table 2), these samples were prepared as tablets having a diameter of about 5/16 inch and tablet weight of about 200 mg, which is in contrast to the commercial dosage form which is significantly larger (1225 mg and larger). The results further show that 2 mg nicotine can be affectively delivered when prepared according to the invention to deliver significantly higher serum concentration (e.g., Cₘₐₓ of about 61 to about 65 ng/ml) in a significantly shorter time period (e.g., tₘₐₓ of about 13 to about 18 min.) via transmucosal delivery as compared to a commercial oral product.

As a further advantage and aspect of the invention, the 2 mg dosage was achieved by a buccal tablet approximately 5/16 inch in diameter, which is relatively and significantly smaller than many conventional lozenge-type products for nicotine. Therefore, the invention affords a more orally comfortable and more convenient packaging options from a manufacturing standpoint.

### Example 5

### Large Scale Preparation of 200 mg Tablet Containing 2 mg Nicotine (from Nicotine Polacrilex)

Large scale preparation of 2 mg nicotine (from nicotine polacrilex) tablets were prepared using a process similar to that described herein above in Example 1. In order to achieve large scale production, the formulation ingredient amounts were adjusted to accommodate the inclusion of microcrystalline cellulose, colloidal silicon dioxide, and flavoring agents. The formulation prepared is set forth in the following table:

TABLE 5
200 mg Tablets containing Nicotine Polacrilex (Large Scale)

| Ingredient: | mg/tablet | % w/w |
|---|---|---|
| Nicotine polacrilex (15%) | 13.33 | 6.67 |
| Mannitol (spray-dried) | 52.42 | 26.21 |
| Sodium bicarbonate | 42.00 | 21.00 |
| Citric acid | 30.00 | 15.00 |
| Silicified microcrystalline cellulose | 25.00 | 12.50 |
| Sodium carbonate | 20.00 | 10.00 |
| Sodium starch glycolate | 10.00 | 5.00 |
| Magnesium stearate | 4.00 | 2.00 |
| Natural and artificial mint flavor | 2.50 | 1.25 |
| Colloidal silicon dioxide | 0.75 | 0.38 |
| Total: | 200.00 mg | 100.0% |

### Nicotine Dosage Form on Holder

In an alternative embodiment to the tablet dosage form described herein above, a larger lozenge-type dosage form can be prepared in which the dosage form formulation of the present invention can be modified into a lozenge affixed or removably attached to a holder or stick. Such dosage form on holder embodiments can be prepared as described in co-pending U.S. Patent Application Ser. Nos. 60/872,177 and 60/872,125, both of which were filed on Dec. 1, 2006—the texts of which are incorporated herein by reference.

According to this particular embodiment, the behavioral aspects of nicotine addiction and smoking are addressed by the presence of the holder or stick, which permits the user to mimic the presence of a cigarette. In this embodiment, the oral dosage form prepared according to the present invention is coupled to one end of the holder, such that the user can maintain the dosage form adjacent to the mucosal tissue and ensure continual positioning adjacent the mucosal tissue by manipulating the holder by hand.

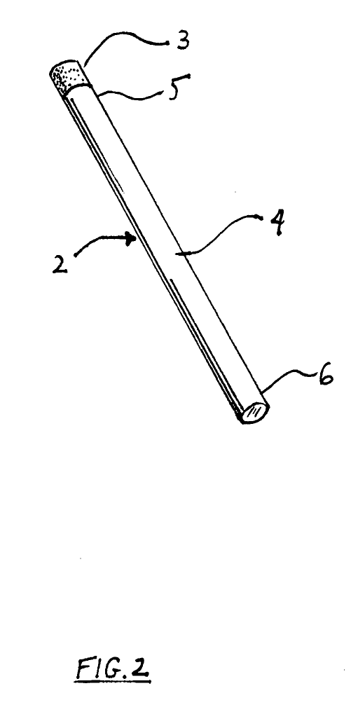

Referring now to FIG. 2, a dosage form on holder system 2 is shown according to one embodiment of the invention. The system 2 can comprise a holder portion 4 and dosage form 3 coupled to the holder portion 4. The holder portion 4 can be dimensioned in a variety of configurations and sizes. In one embodiment and as shown, the holder portion 4 (and dosage form 3) can be constructed according to the typical dimensions of a cigarette. The holder portion 4 can contain two ends—an oral end 5 for placement within the recipient's mouth, and a grasping end 6. The holder portion 4 can be constructed using a variety of materials. Suitable materials include those materials that can afford flexible semi-rigid or rigid structure to facilitate grasping and manipulation of the system by the hand, and such materials can include a variety of plastics and paper materials. The dosage form 3 can be attached to the holder portion 4 a variety of attachment means (not specifically shown), including non-toxic adhesives or glues, coupling structures such as pegs, as an exterior coating, and the like.

As the dosage form prepared according to the invention can be either fixed to a holder or constructed for reversible detachment from a holder, the user can be afforded the option of converting a lollipop-type nicotine delivery system into a free-standing discrete lozenge or dosage form per se according to the user's preferences.

For the particular embodiment wherein the dosage form and holder are reversibly separable to one another, the dosage form contains a reversible coupling structure. The reversible coupling structure can be constructed as: 1) a dosage form structure, e.g., a recess or cavity, which can receive or accommodate an end of the holder; 2) a structure located on the end of a holder, e.g., a friction enhancing texture, which can removably retain the holder in or on the dosage form; or a combination of both such structures.

The holder can further include indicia. Examples of indicia include brandnames, logos, symbols, dosage information, instructions, colors, and the like. Indicia can be applied using various techniques and equipment, such as molding, impressing or embossing techniques, adhesive labeling, and the like, readily available to those skilled in the art.

The holder can further be constructed on the grasping end to include friction-enhancing features, such as tackifiers or pebbling textures. Alternatively and/or in addition to such features, the grasping end can contain finger-specific structures such as tabs and curves.

### INDUSTRIAL APPLICABILITY

The dosage form and composition of the invention can be used for the treatment of nicotine addiction or as a nicotine substitute. Specifically, the invention can be used as part of a nicotine withdrawal therapy program to treat symptoms associated with nicotine cessation, and/or provide a non-tobacco source of nicotine for situations and environments where smoking is prohibited. As a result of then formulation of the invention, the invention affords the ability to manufacture relatively small-sized dosage forms to accomplish effective relatively fast delivery of nicotine to the recipient.

The invention described herein above includes reference to various and specific embodiments and techniques. It will be understood by one skilled in the art, however, that reasonable modifications and variations can be made from said embodiments and techniques without significant departure from either the spirit or scope of the invention as defined by the following claims.

What is claimed is:

1. A solid oral transmucosal dosage form comprising a composition comprising the following ingredients:
a) nicotine or nicotine derivative as an active ingredient;
b) an effervescent couple; and
c) a pH adjusting substance;
said dosage form being formulated for resident placement within a recipient's oral cavity for transmucosal delivery of said nicotine or nicotine derivative across said recipient's oral mucosal tissue.

2. The dosage form according to claim 1, wherein said dosage form composition is in the form of a 200 mg total weight oral buccal transmucosal tablet containing nicotine derivative from about 0.5 mg to about 4.0 mg, said tablet having a diameter of about 5/16 inch.

3. The dosage form according to claim 1, wherein said nicotine derivative is selected from the group consisting of nicotine polacrilex and nicotine bitartrate.

4. The dosage form according to claim 1, wherein said effervescent couple comprises an acid compound and a basic compound and is water soluble or distintegrable and activated by saliva.

5. The dosage form according to claim 4, wherein said acid compound is citric acid and said base compound is sodium bicarbonate.

6. The dosage form according to claim 1, wherein said pH adjusting substance is different from said basic compound of said effervescent couple, and is selected from a carbonate or a phosphate compound.

7. The dosage form according to claim 6, wherein said pH adjusting substance is sodium carbonate.

8. A solid oral transmucosal dosage form comprising the following ingredients:
a) nicotine or nicotine derivative;
b) effervescent couple consisting essentially of citric acid and sodium bicarbonate;
c) a pH adjusting substance comprising sodium carbonate;
d) a filler; and
e) a disintegrating agent;
said dosage form being formulated for resident placement within a recipient's oral cavity for transmucosal delivery of said nicotine or nicotine derivative across said recipient's oral mucosal tissue.

9. The dosage form according to claim 8, wherein said dosage form upon administration achieves a Cₘₐₓ ranging from about 3 ng/ml to about 70 ng/ml and a Tₘₐₓ from about 3 minutes to about 40 minutes respectively.

10. The dosage form according to claim 9, wherein said dosage form achieves a Cₘₐₓ ranging from about 7 ng/ml to about 50 ng/ml and a Tₘₐₓ from about 4 minutes to about 30 minutes, respectively.

11. The dosage form according to claim 10, wherein said dosage form achieves a Cₘₐₓ ranging from about 10 ng/ml to about 25 ng/ml and Tₘₐₓ from about 5 minutes to about 20 minutes, respectively.

12. A method of treating nicotine addiction in a recipient desiring such treatment, said method comprising:
a) providing to said recipient a solid oral transmucosal dosage form comprising:
i) nicotine or nicotine derivative as an active ingredient;
ii) an effervescent couple; and
iii) a pH adjusting substance;
said dosage form being formulated for resident placement within a recipient's oral cavity for transmucosal delivery of said nicotine or nicotine derivative across said recipient's oral mucosal tissue
b) positioning said dosage form within the recipient's oral cavity adjacent to oral mucosal tissue; and
c) permitting said dosage form to reside in such position for a period of time sufficient to permit the nicotine or nicotine derivative to transport across the oral mucosal tissue;
wherein step c) and said dosage form provide a Cₘₐₓ ranging from about 3 ng/ml to about 70 ng/ml and Tₘₐₓ from about 3 minutes to about 40 minutes.

13. An oral transmucosal nicotine delivery system, said system comprising:
a) a solid oral transmucosal dosage form comprising a composition having the following ingredients:
nicotine or nicotine derivative as an active ingredient;
effervescent couple; and
pH adjusting substance;
said dosage form being formulated for placement within a recipient's oral cavity for transmucosal delivery of said nicotine or nicotine derivative across said recipient's oral mucosal tissue; in combination with
b) a holder;
said dosage form being coupled to an end of said holder.

14. The system according to claim 13, wherein said holder is a hand-held stick.

15. The system according to claim 13, wherein said holder and dosage form are constructed for reversible coupling to one another.

16. A method of nicotine substitution comprising:
a) providing a dosage form comprising to a recipient desiring said substitution, said dosage form being a solid oral transmucosal dosage form comprising:
i) nicotine or nicotine derivative as an active ingredient;
ii) an effervescent couple; and
iii) a pH adjusting substance;
said dosage form being formulated for placement within a recipient's oral cavity for transmucosal delivery of said nicotine or nicotine derivative across said recipient's oral mucosal tissue;
b) placing said dosage form within the recipient's oral cavity adjacent to recipient's mucosal tissue; and
c) permitting said dosage form to reside adjacent said mucosal tissue for a period of time sufficient to deliver nicotine across the mucosal tissue.
